(19) United States
(12) Patent Application Publication
(10) Pub. No.: US 2004/0060101 A1
(43) Pub. Date: Apr. 1, 2004

(54) Diving mask

(76) Inventor: Chih Cheng Shiue, Rancho Santa Fe, CA (US)

Correspondence Address: LEONARD TACHNER, A PROFESSIONAL LAW CORPORATION, SUITE 38-E, 17961 SKY PARK CIRCLE, IRVINE, CA, 92614-6364, US

(21) Appl. No.: 10/256,456

(22) Filed: Sep. 27, 2002

(51) Int. Cl. A61F 9/02

(52) U.S. Cl. 2/426

(57) ABSTRACT

A dive mask designed as a compromise of frameless and framed designs. It employs a single frame member or support frame that is smaller and lighter than conventional frame assemblies. The support frame provides a rigid platform for the buckle assembly to be installed. Also because of the support frame, a double lens design is possible. This allows the lenses to be tilted to be closer to the eyes, increasing the field of view. The tilt of each lens is 10-20 degrees from traditional masks. The lenses are thus between 160-140 degrees apart. This improvement in lens placement yields an additional 20-40 degrees of peripheral view.

### BACKGROUND OF THE INVENTION

1. Field of the Invention

The present invention relates generally to the field of dive masks and more particularly to a light frame mask which has the benefits of frameless masks without the disadvantages thereof.

2. Background Art

Traditional masks use frames and subframes to hold a skirt and lens/lenses together. Using tabs on the top and bottom of the subframe, the subframe is locked onto the mainframe. This process also locks the skirt and the lens onto the mainframe. Because of the pressure applied to the lens and skirt, the inside of the mask is waterproof. But two major problems are that the skirt can be accidentally pulled out from frame, and the mask can leak if the seal is not perfect.

The appearance of the mask is set by the shape of the frames. Also the buckle is part of the mainframe and is used to adjust the strap. Because of the need for two frames and the pressure on the skirt and the lens/lenses, there is a required thickness inherent in the mask. This thickness increases the distance between the lenses and the eyes and thus decreases the view of the user.

When the lens in closer to the face, the view angle is greater. A lens that is closer to the face increases the view allowed through the lens. For example a small contact lens provides an unobstructed view to the user, as opposed to a person wearing glasses, which are much larger, but yield a small field of view. The same is true for diving mask lenses, i.e., closer is better.

Diving masks are needed to protect the eyes from the water pressure and from the salt water. The mask is the medium which allows the user to see underwater. The design of the mask and the size of the lenses, greatly affect the view and thus the experience of the person utilizing the mask. Because one can't use contact lenses to dive, the solution is to have the lenses on the mask be as close to the user's eyes as possible. Thus it would be desirable to have a mask which allows the lens or lenses to be closer to the eyes, increasing the available angles of view. The critical distances are from the ridge of the nose between the eyes to just above the cheekbone.

One way to decrease the “volume” of the mask, which directly corresponds to the distance between the eyes and the lens/lenses, is what is called the frameless mask. Because there is no frame, the lens can be placed closer to the face, thus providing a larger angle of view.

The entire center structure of the frameless mask is made out of silicon. Using a bonding agent, the lens/lenses are glued onto the silicone skirt while the skirt is being injected. This produces an extremely strong bond between the lens and the skirt. There are two methods of applying this bonding agent, 1) applying it to the lens, placing the lens into the mold and then injecting the silicon into the mold, to create the mask, or 2) applying the bonding agent directly into the silicone and then injecting this mixture into the mold of the mask, with the lens in place within. Once this process is complete, the silicon skirt is wrapped around the lens, creating the entire center structure of a traditional mask. All other parts, i.e., buckle assembly etc., are made of plastic like PC, PC/ABS, ABS, etc.

Unfortunately, frameless masks have a number of drawbacks:

1) Since the buckle is not set on a rigid part of the mask, this can cause problems. Because it is installed on the skirt, when the strap is tightened by using the buckle, the skirt is often deformed and creased. This will cause the mask to be uncomfortable and also often leak.

2) Frameless masks must use a single lens. They use one piece of glass because without a frame, there is a need for support for the center of the mask. Since the entire mask is made of silicon (other than the buckle assembly), the lens takes the place of the plastic frame in providing this support. This is a drawback because in a single lens mask, the glass must be set further from the face than on double lens masks. This is due to the single lens need to be higher than the ridge of the user's nose. This causes the view to be smaller, which must be compensated for by making the lens larger.

3) Because of the need to support the glass and buckle assembly, a frameless mask must use silicone to take the place of the frame. Thus 20%-25% more silicon is used than in manufacturing a traditional mask. This causes a substantial increase in cost because silicone is 3-4 times more expensive than other plastics such as PC, PC/ABS, ABS, which is what traditional masks frames are made of. Another problem that arises from the use of additional silicone is that it takes additional time for the mask to cool down once the injection process is completed. Thus it takes longer to produce each mask, increasing the cost.

4) Because there is no frame, the entire appearance or style of the mask depends on the shape of the lens. Because the mask relies on the lens for support and the lens is elevated, it is easily broken. Also the point where the glass and silicone meet is in danger of being cut and thus ruining the mask.

### SUMMARY OF THE INVENTION

The mask of the present invention is a combination of frameless and framed designs. It employs a single frame member or support frame that is smaller and lighter than conventional frame assemblies. First the silicon is injected to make the skirt, with the lens installed in the mold, such as in the frameless masks. Because of this process, the inside of the mask is already waterproof, because the lens is affixed to the skirt. This also resolves the problems of leakage and of the skirt being pulled from the frame of a traditional mask.

After the injection process, a small support frame is assembled over the silicone mask. The function of the support frame is to protect the mask from dropping damage, and to protect the points where the glass and skirt are glued. This support frame also provides a space where the buckle assembly can be installed on a rigid location.

The support frame has a “U” shaped indent so that the silicone and lens mask can be installed. Once the mask is installed into the support frame, a locking mechanism, located either in the center or on the left and right sides of the mask, locks the support frame onto the mask.

Because of the support frame, not as much silicone is needed to support the mask. This resolves the increase of production time that is a drawback with frameless masks. Also the support frame is smaller than the traditional mainframe/subframe combination because it does not need the additional material to lock the skirt onto the glass. The skirt and the glass are already affixed together, thus pressure is not needed to make the skirt waterproof or to keep the skirt from slipping out of the frame. Thus the inventive mask provides the low volume of frameless masks, with the protection of a rigid frame. The mask is also simpler, both in design and in manufacturing.

Another advantage of the support frame is that it provides a rigid platform for the buckle assembly to be installed. Thus where the tightening of the strap would otherwise crease the skirt and thus cause discomfort and leakage in a frameless mask, this is avoided in the inventive mask. Also because of the support frame, a double lens design is possible. This allows the lenses to be closer to the eyes, increasing the field of view.

During the injection process, the lenses of the preferred embodiment are held at less than 180 degrees apart when viewed from the user's point of view. Thus the lenses are closer to the eyes, when compared to frameless and traditional masks. To lock down this angle on the skirt and lenses, the support frame has this angle built in either by arching the frame from side to side or by angling one side relative to the other. The goal of this process is to place the lenses as close to the eyes as possible. The inventive mask thus decreases the distance between the lenses and the eyes by up to 70%. Because of the reduced distance, the field of view is larger. The preferred lenses are flat or planar to minimize optical aberration and distortion.

The tilt of each lens is 10-20 degrees from traditional masks. The lenses are thus between 160-140 degrees apart. This improvement in lens placement yields an additional 20-40 degrees of peripheral view. This is a significant improvement over traditional masks, which usually have a field of view of 130-150 degrees.

Frameless masks have solved problems inherent in traditional masks. Traditional masks are large because of the need for support of the lens, the skirt, and the locking devices of the subframe and the mainframe. The inventive mask also solves the problem of the frameless masks by adding a support frame around the lens/silicone combination. Thus the problem of the tearing of the skirt from the lens is solved. The problem of the single lens is solved. The problem of the buckle assembly creasing the skirt is solved. The problem of the easy breaking of the glass is solved. Thus, the inventive mask, improves on the frameless mask's shortcomings, while not losing many of its benefits.

### BRIEF DESCRIPTION OF THE DRAWINGS

The aforementioned objects and advantages of the present invention, as well as additional objects and advantages thereof, will be more fully understood herein after as a result of a detailed description of a preferred embodiment when taken in conjunction with the following drawings in which:

FIG. 1 is prior art drawing of a conventional dive mask;

FIG. 2 is an exploded view of the mask of FIG. 1;

FIG. 3 is a cross-sectional view taken along lines A-A of FIG. 1;

FIG. 4 is a prior art drawing of a frameless dive mask;

FIG. 5 is an exploded view of the mask of FIG. 4

FIG. 6 is a cross-sectional view taken along lines C-C of FIG. 4;

FIG. 7 is a cross-sectional view taken along lines D-D of FIG. 4;

FIG. 8 is a view of a preferred embodiment of the present invention;

FIG. 9 is an exploded view of the mask of FIG. 8

FIG. 10 is a cross-sectional view of the mask of FIG. 8 similar to that of FIG. 6;

FIG. 11 is a cross-sectional view of the mask of FIG. 8 similar to that of FIG. 7;

FIG. 12 is an enlarged partial view of the conventional mask;

FIG. 13 is an enlarged partial view of the frameless mask;

FIG. 14 is an enlarged partial view of the preferred embodiment of the invention;

FIG. 15 is a simplified illustration used to explain the field of view of a conventional dual lens configuration; and

FIG. 16 is a simplified illustration used to explain the expanded field of view of a tilted lens configuration of the invention.

### DETAILED DESCRIPTION OF A PREFERRED EMBODIMENT

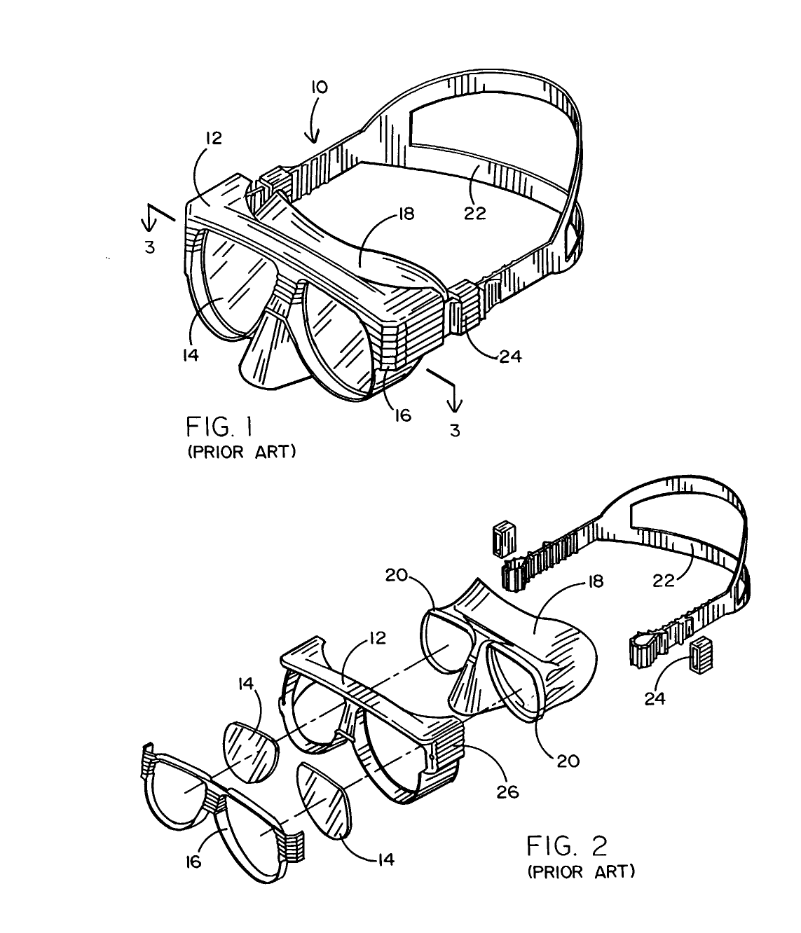

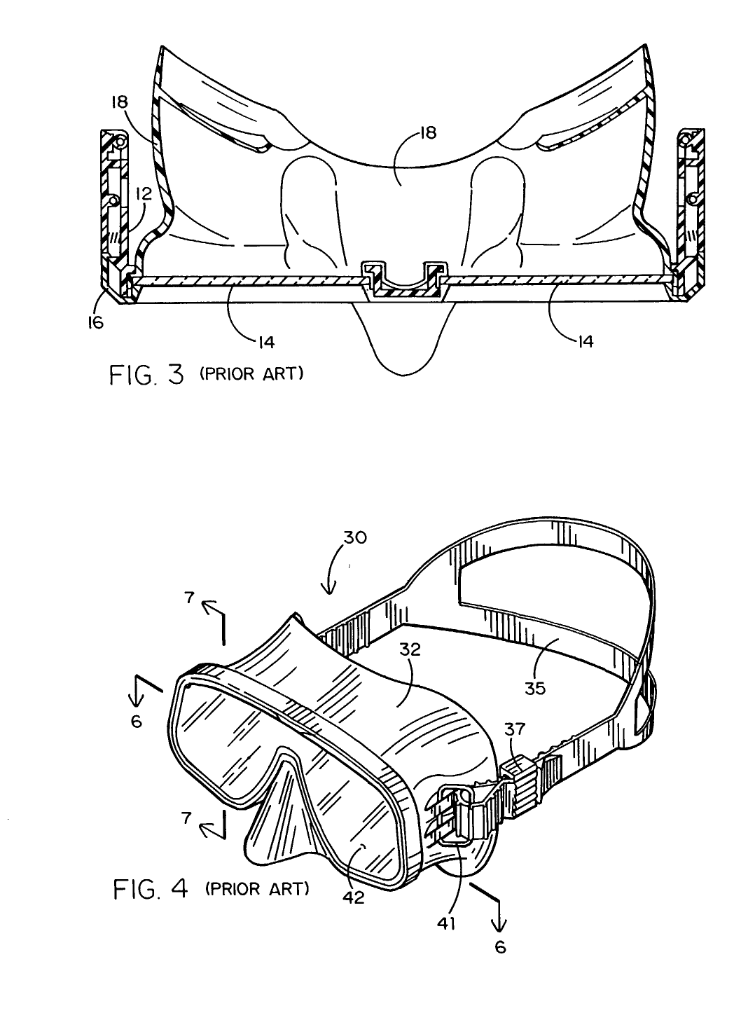

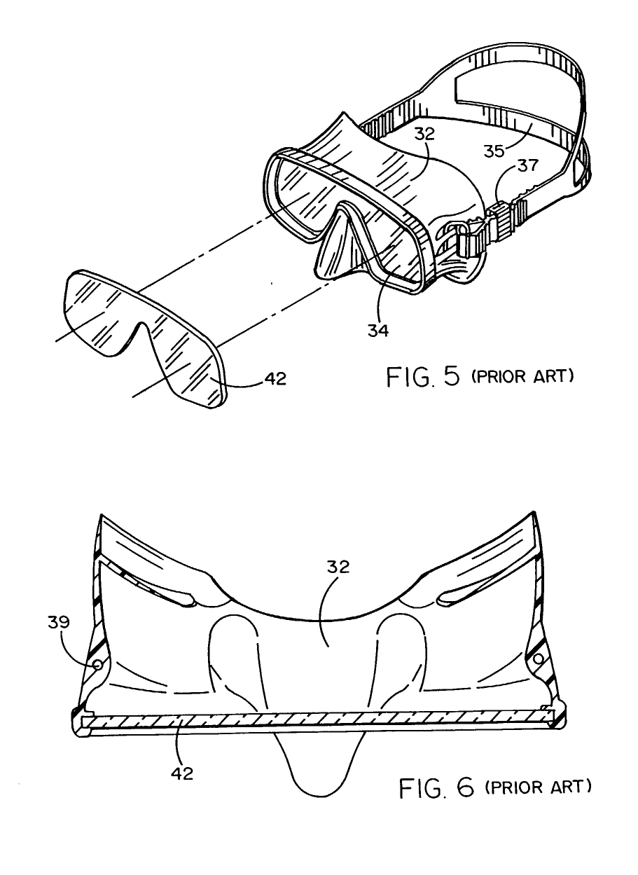

Referring to the accompanying figures and initially to prior art FIGS. 1-5, it will be seen for purposes of comparison with the present invention, that there is shown a conventional dive mask 10. Mask 10 comprises a main frame 12, lenses 14, a subframe 16, a silicone skirt 18 having lens receiving apertures 20, a strap 22 and strap holder 24 designed to cooperate with a buckle 26 on main frame 12. It will be noted that conventional mask 10 has a pair of lenses 14 which are situated at 180 degrees to one another as seen in FIG. 3. It will also be observed that the conventional mask has both a main frame and a subframe, thereby suffering the disadvantages noted above.

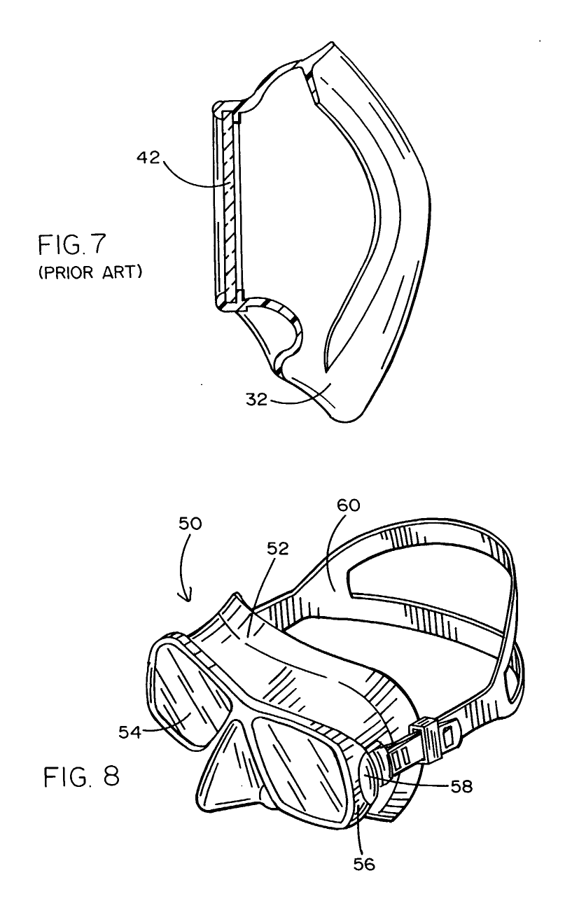

Referring now to prior art FIGS. 4-7, it will be seen for purposes of comparison with the present invention, that there is shown a so-called frameless dive mask 30. Mask 30 comprises a skirt 32 having a lens aperture 34, a strap 35 having a buckle 37 and a lens 42. As seen in FIG. 6, the skirt 32 has a pair of holes 39 to receive a buckle attachment ring 41. It will be observed that the mask 30 suffers the disadvantages noted above, namely, the required single large lens, the direct buckle connection to the silicone skirt and the considerably larger volume of silicone as compared to both a conventional mask and the present invention as will be seen hereinafter.

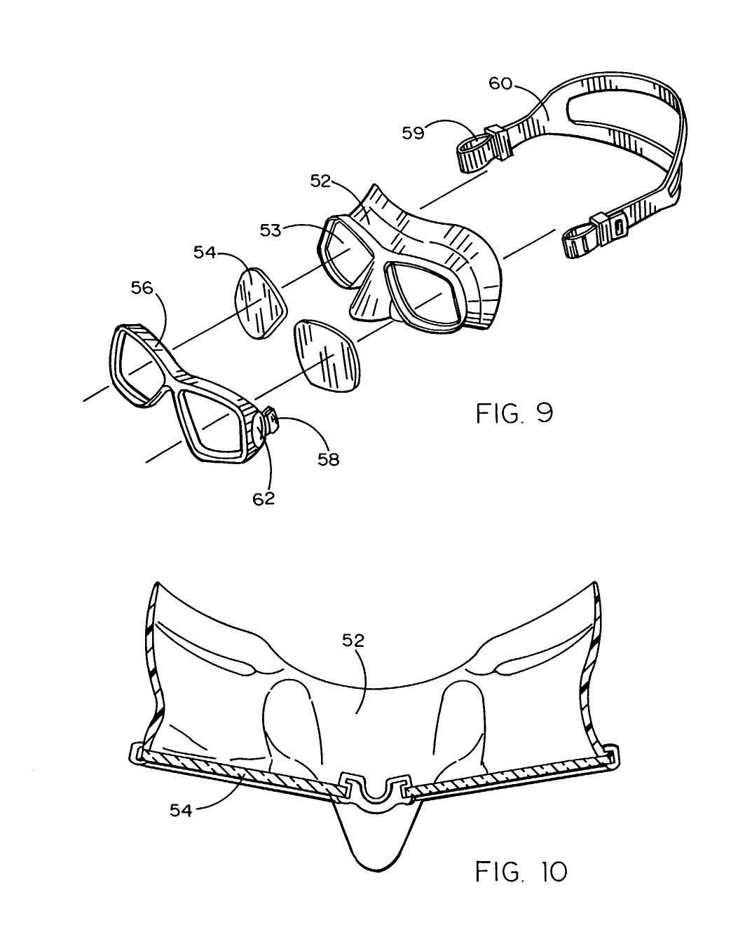

Referring now to FIGS. 8-11 it will be seen that a preferred embodiment 50 of the present invention comprises a skirt 52 having lens apertures 53, a pair of lenses 54 and a unitary light frame 56. Mask 50 also has a strap 60 having a pair of buckle loops 59 configured to mate buckles 58 connected to buckle receptacles 62 which are affixed to or integral with light frame 56. The double lens structure of the preferred embodiment is typically not found in the frameless mask because the latter must rely on the structural integrity of a large single lens to lend rigidity to the frameless structure. It is more like the conventional dual lens structure of the conventional mask, but nevertheless differs therefrom in a very significant way. This difference may be observed by comparing the illustration of FIGS. 15 and 16. The lenses of a conventional mask are, as shown in FIG. 15, both equidistant from the eye across the entire length of each lens and are co-linear or 180 degrees apart. On the other hand, the lenses of the present invention are preferably tilted so that the respective ends (d₂ end) of the lenses that are furthest from the nose area (i.e., between the lenses) are closer to the eyes so that the lenses are at an angle less than 180 degrees (preferably by 10-20 degrees). The result is a significantly larger field of view as described above. Light frame 56 is preferably arched or angled from side to side to facilitate installation of lenses 54 in a tilted configuration as shown best in FIG. 10.

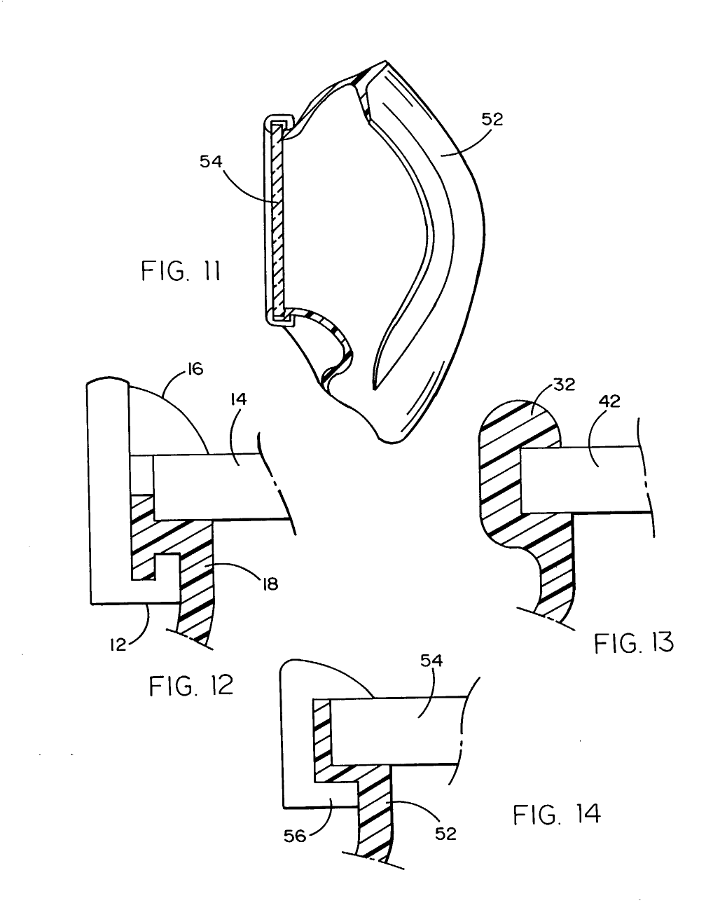

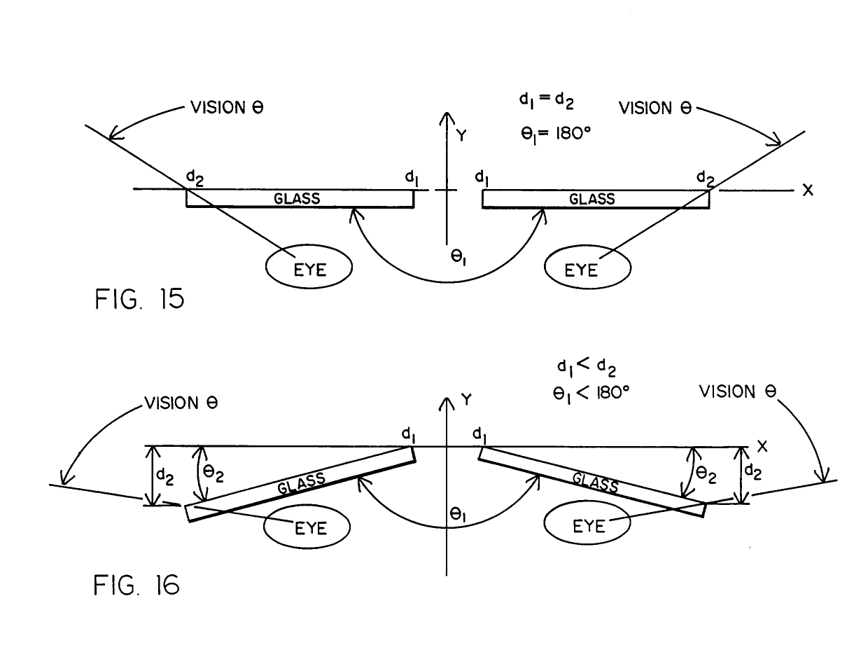

Finally, the contrast between mask structures disclosed herein (i.e., conventional, frameless and the present invention), is most evident in the details of lens retention which are shown in FIGS. 12 to 14. FIG. 12 illustrates that in a conventional mask, lens 14 is secured by both a large main frame 12 and smaller subframe 16 with the skirt 18 only partially engaging the lens. FIG. 13 illustrates that in a frameless mask, lens 42 is fully engaged by thick skirt 32. FIG. 14 illustrates for the preferred embodiment of the inventive mask that lens 54 is secured by light frame 56 and reduced thickness skirt 52 which extends over the entire edge of the lens. Thus frame 56 is locked to both the lens 54 and the skirt 52.

It will now be understood that the dive mask of the present invention has many of the advantages of frameless masks as compared to conventional masks, but avoids disadvantages of frameless masks as well. It will also be understood that although a preferred embodiment has been disclosed herein, the disclosure will now inspire those having skill in the relevant art to perceive various modifications and additions. However, the invention described herein contemplates many such variations and therefore is not limited to the particular described embodiment. The invention hereof is limited only by the appended claims and their equivalents.

### I claim:

1. A dive mask having a single frame member, the mask comprising:
a skirt made of a flexible material and having at least one less aperture;
at least one planar lens secured within said at least one lens aperture;
a unitary frame affixed to said skirt and to said at least one lens and having an arched exterior surface from one side of said mask to another side of said mask; said frame having opposed buckle receptacles adjacent said respective sides; and
a strap terminating in opposed buckles, said buckles being received in said buckle receptacles.

2. The dive mask recited in claim 1 wherein said skirt has two lens apertures, and wherein said mask comprises two distinct planar lenses, one respective lens for view by each respective eye of a user of said mask.

3. The dive mask recited in claim 2 wherein said two distinct planar lenses are tilted relative to one another to an angle less than 180 degrees.

4. The dive mask recited in claim 1 wherein said unitary frame is locked to both said skirt and said at least one lens.

5. The dive mask recited in claim 1 wherein said skirt is molded to said at least one lens.

6. A dive mask having a single frame member, the mask comprising:
a skirt made of a flexible material and having at least one lens aperture;
at least one planar lens secured within said at least one lens aperture;
a unitary frame affixed to said skirt and to said at least one planar lens and having an angled exterior surface from one side of said mask to another side of said mask; said frame having opposed buckle receptacles adjacent said respective sides; and
a strap terminating in opposed buckles, said buckles being received in said buckle receptacles.

7. The dive mask recited in claim 6 wherein said skirt has two lens apertures, and wherein said mask comprises two distinct planar lenses, one respective lens for view by each respective eye of a user of said mask.

8. The dive mask recited in claim 7 wherein said two distinct planar lenses are tilted relative to one another to an angle less than 180 degrees.

9. The dive mask recited in claim 6 wherein said unitary frame is locked to both said skirt and said at least one lens.

10. The dive mask recited in claim 6 wherein said skirt is molded to said at least one lens.

11. A dive mask comprising:
a flexible skirt having a pair of lens apertures;
a pair of planar lenses secured in said lens apertures; and
at least one rigid frame affixed to said skirt and to said lenses;
said lenses being symmetrically tilted from side to side so that the angle between said lenses is less than 180 degrees.

12. The dive mask recited in claim 11 wherein said skirt has a nose piece between said lens apertures, and wherein each said lens is further from a wearer's eye adjacent said nose piece and nearer to a wearer's eye further from said nose piece.

13. The dive mask recited in claim 11 wherein said at least one rigid frame comprises a unitary frame member that is locked to said skirt and to said lenses.

14. The dive mask recited in claim 11 wherein said skirt is molded to said lenses.
